(19) United States
(12) Patent Application Publication
(10) Pub. No.: US 2021/0228918 A1
(43) Pub. Date: Jul. 29, 2021

(54) SUPPLIED AIR RESPIRATOR

(71) Applicant: RPB Safety, LLC, Royal Oak, MI (US)

(72) Inventors: Garth Ivory, Bloomfield Hills, MI (US); William C. Hamill, Troy, MI (US); Edward S.M. Williams, Christchurch (NZ); Alan J. Gerrard, Christchurch (NZ); Samuel M. Kelly, Christchurch (NZ)

(21) Appl. No.: 17/156,448

(22) Filed: Jan. 22, 2021

Related U.S. Application Data
(60) Provisional application No. 62/965,084, filed on Jan. 23, 2020.

(51) Int. Cl. A62B 9/00; G06F 1/3212; A61M 16/00

(52) U.S. Cl. CPC A62B 9/006; A61M 2016/0033; A61M 16/0003; G06F 1/3212

(57) ABSTRACT

A supplied air respirator having an airflow path to supply supplied air to a user. The respirator includes a control unit having a sensor located in the airflow path that communicates with a CPU. The CPU also communicates with an indicator that can provide either tactile, audible or visual status indications. One or any combination of these can be used. A power source is provided for powering the CPU and the indicators. The. CPU compares a sensor signal from the sensor with a predetermined operating flow rate and a desired flow rate and indicates to the user the status of the respirator. The CPU continually communicates with said sensor and does not shut down.

### CROSS REFERENCE TO RELATED APPLICATION

This application claims priority to U.S. Provisional Application No. 62/965,084 filed Jan. 23, 2020, which is incorporated herein by reference in its entirety.

### BACKGROUND OF THE INVENTION

### 1. Field of the Invention

The present invention relates to a supplied air respirator and more particularly to a supplied air respirator with a controller that signals various modes of operation of the supplied air respirator. The controller has an indicator that can signal a user when for example the respirator is operational, when the airflow is at a desired level for use, when the respirator is in standby mode and the battery life of the controller.

### 2. Description of the Prior Art

Warning devices for compressed air systems are generally known in the art for alerting the user when their flow rate is below a recommended level. The recommended level is often defined by the governing occupational health and safety agency. For example, the European standard BS EN 14594-2018, requires the respirator to have continuous flow of 1601/min.

Typically, warning devices do not have an electrical power source. This limits both the indication method for the warning device and the information that the warning device can provide. Examples of current indicating devices are described in EP-1038 553, DE-A-30 32 371, GB-A-2 130 893, U.S. Pat. No. 4,765,326, and in EP-A-0 349 191 and 0 602 847.

An example of a respiratory protective equipment is disclosed in U.S. Pat. No. 6,615,828B1. The warning device contains a flow detecting orifice and uses a pressure-responsive indicator device that alerts the user when the airflow is below a predetermined value. The indicator is a ball that floats in a tube to indicate the airflow rate. This is similar to known systems in that the indicator is not electrically powered. They are mechanical in operation. This is particularly true of supplied air respirators which typically do not have electric power. Also, none of the prior art provides a tactile response which can be very important in highly noisy environments.

### SUMMARY OF THE INVENTION

The present invention provides the user of a supplied air respirator with an indicator that indicates when the flow rate of air traveling to their breathing zone is below a recommended threshold by tactile, audible or visual indications or a combination of these. The device contains an electrical power source that powers the warning device. The device includes a differential pressure sensor, which measures the airflow across a pressure drop, a CPU to control the indicator and a vibration motor that is used as an indicator to the user of relevant information. A barometric pressure sensor compensates for changes in atmospheric pressure.

The differential pressure sensor is in fluid communication with the breathable air traveling across the pressure drop orifice. The sensor takes intermittent readings that are used to calculate the flowrate. The warning device has two main functions, operating mode and standby mode. In the operating mode, the warning device takes regular measurements and within a specific response time communicates to the user of the supplied air respirator. In the standby mode, the warning device conserves the use of the electrical power source and has longer periods between measurements. If a reading above the operating threshold is recorded, the warning device transitions to operating mode.

In the operating mode, there are two thresholds, the operating threshold and the desired threshold. The operating mode includes an indicating mode that alerts the user if they are not receiving enough airflow. The operating threshold and recommended threshold are easily changed and can be adjusted depending on the requirements of the governing body or requirements of the end user. Different settings can also be selected to meet the requirements of different supplied air respirators. In other words, the warning device can be tuned according to specific market requirements.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 illustrates the supplied air respirator of the present invention.

FIG. 2 illustrates a cross sectional view of the air supply tube of the air respirator with the control unit of the present invention.

FIG. 3 is a flow chart illustrating the functions of the control unit of the present invention.

### DETAILED DESCRIPTION OF THE INVENTION

The supplied air respirator of the present invention is shown generally at 10. The respirator 10 includes a supplied air source 12, an air path or airflow tube 14 and a respirator headpiece 16. As will be appreciated by those of ordinary skill in the art, the supplied air source 12 provides pressurized air through the air path 14 to create positive pressure in the headpiece 16. It should be appreciated that the headpiece can take many different forms for example, a helmet, a visor or a hood.

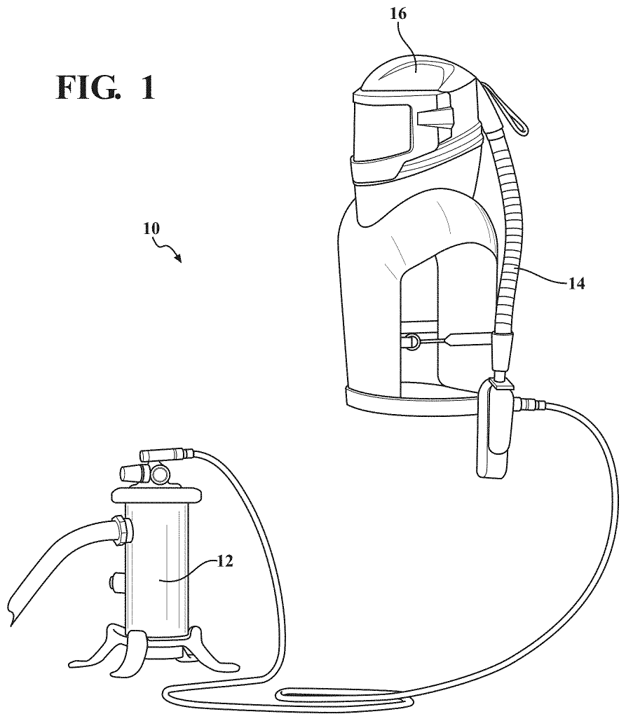

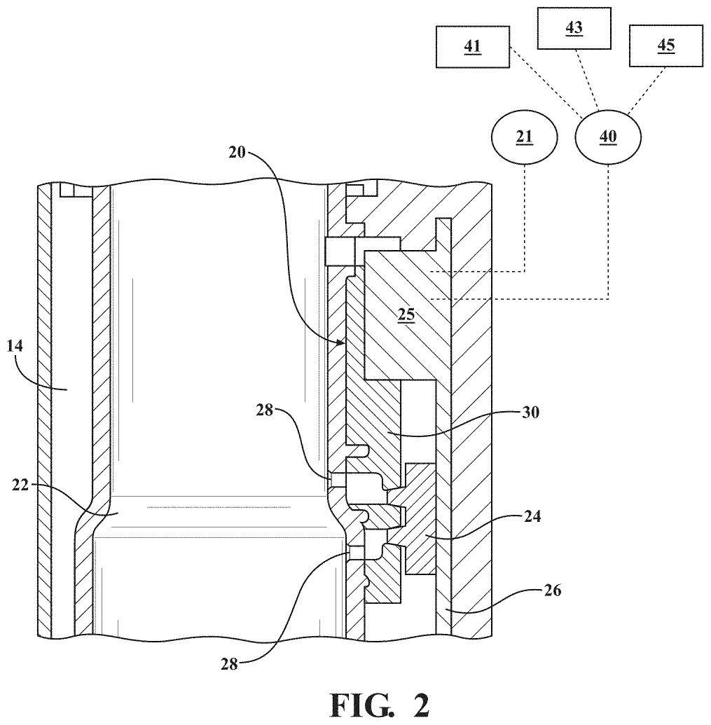

With reference to FIGS. 1 and 2, the respirator 10 has a control unit shown generally at 20 that provides a signal to the user of the operating modes of the respirator 10. The signal can be tactile, such as a vibration, auditory, such as a buzzer, or visual, such as a light or a combination of these.

The respirator 10 has this capability because the control unit 20 which acts as a warning device is electrically powered by a power source 21. In the disclosed embodiment, the power source 21 is a battery. The power source 21 and control system enhances both the signal method and the information that can be provided. It should be appreciated that tactile is important in many work environments because the noise level can make hearing an audible warning difficult.

The control unit 20 of the present invention monitors the airflow to the user by determining the pressure differential across a pressure drop 22. In the disclosed embodiment, the control unit 20 is positioned adjacent to the pressure drop 22 which is positioned within the airflow tube 14 that supplies air to the user. A differential pressure sensor 24 is mounted to the printed circuit board 26 and receives the pressure from the sensor ports 28. In the disclosed embodiment, the sensor compensates for change in atmospheric pressure. This increases the accuracy of the control unit 20 and the indications provided.

Sensor ports 28 are positioned before and after the pressure drop 22. In the disclosure the pressure drop 22 is shown as a venturi. A seal and support 30 are provided to seal the ports and support the sensor 24. By determining the pressure differential across the pressure drop 22, the flow can be determined by an onboard CPU 25. The sensor 24 measures the pressure drop at the pressure drop 22 arranged in the discharged air. Discharged air is any air that has travelled through a flow valve or supplied air respirator.

The pressure sensor 24 compensates for change in atmospheric pressure and the CPU 25 determines the volume of flow based on the measured pressure differential. The CPU 25 can store the discharged airflow rate, discharged air temperature, and ambient temperature at any given measuring interval.

When the flow drops below a predetermined desired threshold, the recommended desired flow rate, the sensor 24 sends a signal to the CPU 25, which then sends a signal to the indicator 40. The indicator 40 may give a tactile response, such as causing a motor to vibrate. The vibration motor 41 is secured to the supplied air respirator and operates when prompted by the CPU 25. The motor 41 can be directly attached to the control unit 20, along the tube 14 or in the headpiece 16.

The indicator 40 may also use an auditory response 43 through for example a piezo buzzer as an indicator and sounds when prompted by the CPU 25. By way of example, the piezo buzzer emits sounds between 2000-4000 Hz. The indicator 40 could also be visual 45, such as for example one or more LED lights. The indicator 40 can also provide a combination of multiple responses, for example, the device can indicate with a piezo buzzer, a vibration motor and a light or any other combination of these as desired.

The control unit 20 has a set lower limit known as the operating threshold. The control unit alarms down to the predetermined operating threshold but it does not alarm after the supplied air respirator 10 airflow rate is below this set value. The CPU 25 is unable to be turned off, instead a standby function or power save mode is activated when the airflow rate is below the operating threshold.

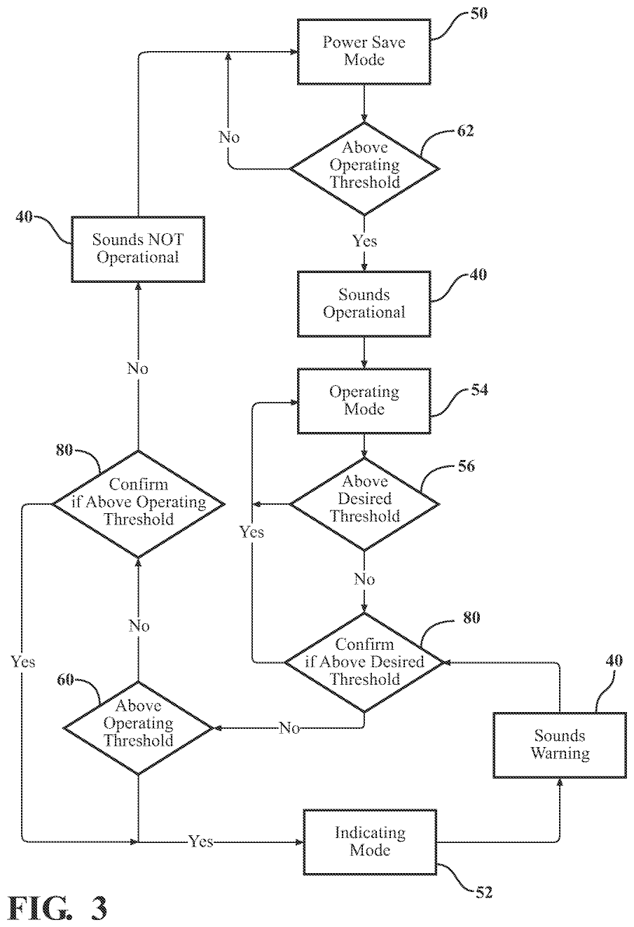

With respect to FIG. 3, a flow chart of the operation of the control unit is illustrated. The Flow Chart shown in FIG. 3 is merely an example visualization of the operation of the control unit and does not necessarily represent the logic structure of the code the CPU 25 is programmed with. The respirator 10 starts in the power save mode 50. The CPU 25 is on but it is conserving energy by only communicating with the sensor 24 intermittently, at measuring time intervals that are longer than the other time intervals used by the CPU 25. The respirator 10 stays in the power saving mode 50 until the value detected by the sensor and communicated to the CPU 25 is above the operating threshold 62. This operating threshold 62 indicates that supplied air is being provided to respirator 10.

When the sensor 24 communicates with the CPU 25 a pressure differential that converts to a flow of for example over 50 slpm, the operating threshold 62, the control unit 20 is in the operating mode 54. If the flow is under the desired threshold 56, for example 170 slpm, the control unit 20 is in the indicating mode 52. The indicating mode 52 is when the indicator 40 sounds a unique warning that alerts the user that they are not receiving enough air. The CPU 25 and sensor 24 communicate in shorter intervals at the indicating mode 52 when in operating mode 54. The CPU 25 can instruct the indicator 40 to indicate that the control unit 20 is in the indicating mode 52. It will be appreciated by those of ordinary skill in the art that the value setting to reach the operating mode 54, the desired and operating thresholds 56 and 62 and the indicating mode can be changed as desired.

When the sensor 24 communicates with the CPU 25 a pressure differential that converts to a flow of for example above 170 slpm, the control unit 20 has reached the desired threshold 56, in the operating mode 54. Above the desired threshold 56, the desired flow is being supplied to the user. The CPU 25 enters a loop to continually communicate with the sensor 24 to confirm that the flow is above the desired flow of the desired threshold 56. The indicator 40 can notify the user of the status by vibrating at a different vibration rate, sounding a different sound or providing a green light for example. It will be appreciated by those of ordinary skill in the art that the values to reach the modes and thresholds can be changed as desired.

When the sensor 24 communicates with the CPU 25 a drop in the flow rate under the desired threshold 56, but above the operating threshold 62, the CPU 25 sends instructions to the indicator 40 to signal the desired flow has been lost. If the flow drops below 50 slpm the control unit enters the power save mode 50. Again, an indication of this can be given by for example a slowing vibration, a lowering of the audible volume or a yellow to red light. If the flow stays above 50 slpm, the CPU enters a second confirming loop 60. If the confirming loop determines a flow rate above 170 slpm, the control unit is in the desired threshold 62 and the control unit 10 can indicate the status. Both confirming loops 56 and 60 can have an additional check by a second confirming condition 80 to avoid unnecessary indications or mode changes in case of single false reading of the sensor.

In the disclosed embodiment, the control unit 10 can be a stand-alone device and used with various commercially available respirators. In other words, it can be sold as a unit and attached to existing respirators. The control unit 10 has a housing that encases all the components. In the disclosed embodiment, the casing is sealed from the outside environment, with the indicators being inside the casing.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

What is claimed is:

1. A supplied air respirator comprising:
an airflow path to supply supplied air to a user;
a sensor in fluid communication with said airflow path;
an indicator;
a CPU communicating with said sensor and said indicator;
a power source for powering said CPU;
said CPU comparing a sensor signal from said sensor with a predetermined operating threshold and a desired threshold;
said respirator entering a power save mode when said sensor signal is below said operating threshold;
said CPU communicating with said indicator to send an alert when said sensor signal is above the operating threshold and below said desired threshold;
said CPU continually communicating with said sensor and not shutting down.

2. The supplied air respirator of claim 1, wherein said CPU communicates with said indicator to send a signal when said operating threshold has been reached.

3. The supplied air respirator of claim 1, wherein said CPU communicates with said indicator to send a signal when said sensor signal is at or above said desired threshold.

4. The supplied air respirator of claim 3, wherein said CPU and said sensor communicate less when in said power save mode.

5. The supplied air respirator of claim 1, wherein said power source is a battery.

6. The supplied air respirator of claim 1, wherein said CPU monitors said battery and communicates with said indicator to indicate when the battery capacity is below the low battery threshold.

7. The supplied air respirator of claim 4, wherein said signals are tactile, auditory or visual or a combination thereof.

8. A control unit for a supplied air respirator; said control unit comprising:
a flow path through which air flows to a user;
a pressure sensor in fluid communication with said flow path;
an indicator to notify a user regarding the operation of said supplied air respirator;
a CPU communicating with said pressure sensor and said indicator;
a power source for powering said CPU;
said sensor determining a pressure differential within said flow path, said sensor communicating said pressure differential to said CPU, said CPU comparing said pressure differential to a predetermined operating rate, if said pressure differential is below said predetermined operating rate, said CPU communicating with said control unit to enter power save mode;
said CPU comparing said pressure differential to said predetermined operating rate, if said pressure differential is above said predetermined operating rate, said CPU communicating with said indicator to signal said control system is operational;
said CPU comparing said pressure differential to a predetermined desired operating rate; if said pressure differential is between said desired operating rate and said predetermined operating rate said CPU communicating with said indicator to signal said control unit is operational but below desired operating rate.

9. The supplied air respirator of claim 8, wherein said CPU and said sensor communicate less when in said power save mode.

10. The supplied air respirator of claim 8, wherein said CPU monitors said battery and communicates with said indicator to indicate when the battery capacity is below the low battery threshold.

11. The supplied air respirator of claim 8, wherein said signals are tactile, auditory or visual or a combination thereof.

12. A supplied-air respirator comprising:
an airflow path for supplying air to a user;
a pressure sensor in fluid communication with said airflow path;
a CPU unit, said CPU unit receiving pressure readings from said pressure sensor;
an indicator;
said sensor determining a pressure differential inside said airflow path, said sensor communicating said pressure differential to said CPU, said CPU converting said pressure differential to a flow rate;
said CPU comparing said flow rate to a predetermined desired flow rate; if said flow rate is below said predetermined desired flow rate, said CPU communicating with said indicator to issue a first signal that said predetermined desired flow rate has not been met;
said CPU comparing said flow rate to a predetermined operating flow rate; if said flow rate is below said predetermined operating flow rate, said CPU placing said respirator in power save mode.

13. The supplied air respirator of claim 12, wherein said CPU and said sensor communicate less when in said power save mode.

14. The supplied air respirator of claim 12, wherein said CPU monitors said battery and communicates with said indicator to indicate when the battery capacity is less than 15% and when said battery capacity is depleted.

15. The supplied air respirator of claim 4, wherein said signals are tactile, auditory or visual or a combination thereof.
